(19) United States
(12) Patent Application Publication
(10) Pub. No.: US 2025/0127393 A1
(43) Pub. Date: Apr. 24, 2025

(54) PERFORMING A CONTACT LENS CHECK

(71) Applicant: Alcon Inc., Fribourg (CH)

(72) Inventor: Tobias Jura Rapoport, Berlin (DE)

(21) Appl. No.: 18/914,349

(22) Filed: Oct. 14, 2024

Related U.S. Application Data
(60) Provisional application No. 63/592,691, filed on Oct. 24, 2023.

(51) Int. Cl. A61B 3/10; A61B 3/00; A61B 3/107; A61B 3/117; A61B 3/14

(52) U.S. Cl. CPC A61B 3/1015; A61B 3/0041; A61B 3/102; A61B 3/107; A61B 3/117; A61B 3/14

(57) ABSTRACT

In certain embodiments, an ophthalmic system for examining the eye of a patient includes a detector and computer. The detector detects waves reflected from the eye, and the waves convey information about the eye. The detector then generates a detector signal that communicates the information about the eye. The computer performs a contact lens check by determining a characteristic from the eye information indicating the potential presence of a contact lens. The computer generates a notification warning of the potential presence of a contact lens and instructs an output device to provide the notification.

### TECHNICAL FIELD

The present disclosure relates generally to ophthalmic measurement devices, and more particularly to performing a contact lens check for an ophthalmic measurement device.

### BACKGROUND

An eye examination may involve a variety of measurements of the eye. For example, ocular biometry measures the anatomy of the eye, such as the axial length (AL) and anterior chamber depth (ACD), to determine the segments of the eye. These measurements are crucial for selection of the correct intraocular lens (IOL) power to achieve the desired refractive outcome. As another example, keratometry describes the shape of the anterior surface of the cornea, which can be used to design laser refractive treatment. These and other types of measurements should be accurate to achieve the best visual outcomes for patients.

### BRIEF SUMMARY

In certain embodiments, an ophthalmic system for examining an eye of a patient includes a detector and a computer. The detector detects waves reflected from the eye, where the waves convey information about the eye. The detector then generates a detector signal that communicates the information about the eye. The computer performs a contact lens check by receiving the eye information via the detector signal. The computer then determines from the information a characteristic indicating the potential presence of a contact lens, generates a notification warning of the potential presence of a contact lens, and instructs an output device to provide the notification.

Embodiments may include none, one, some, or all of the following features:

The detector is an ocular biometer, and the eye information describes segmentation of the anterior portion of the eye. The computer determines the characteristic by detecting an additional segment anterior to the cornea that represents the contact lens.

The detector is a keratometer, and the eye information describes the axial length of the eye. The computer determines the characteristic by detecting an increase in the axial length relative to an axial length measurement of the eye without a contact lens.

The detector is an optical coherence tomography (OCT) device, and the eye information describes surfaces of the eye. The computer determines the characteristic by detecting an additional surface anterior to the cornea that represents the contact lens.

The detector is a camera, and the eye information describes one or more Purkinje images of the eye. The computer determines the characteristic by detecting a Purkinje image resulting from the presence of a contact lens.

The eye information describes surfaces of the eye. The computer determines the characteristic by detecting an additional surface anterior to the cornea.

The computer performs the contact lens check automatically for each patient.

The computer performs the contact lens check in response to a request to perform the contact lens check.

The output device is a display device, and the notification is a visual indicator warning of the potential presence of a contact lens.

The output device is a speaker, and the notification is an auditory indicator warning of the potential presence of a contact lens.

In certain embodiments, a method for examining an eye of a patient includes detecting, by a detector, waves reflected from the eye of the patient, where the waves convey information about the eye. A detector signal that communicates the information about the eye is generated by the detector. A contact lens check is performed by a computer by: receiving the information about the eye via the detector signal; determining from the information about the eye a characteristic indicating a potential presence of a contact lens; generating a notification warning of the potential presence of a contact lens; and instructing an output device to provide the notification warning of the potential presence of a contact lens.

Embodiments may include none, one, some, or all of the following features:

The detector is an ocular biometer, and the eye information describes segmentation of the anterior portion of the eye. The method determines the characteristic by detecting an additional segment anterior to the cornea that represents the contact lens.

The detector is a keratometer, and the eye information describes the axial length of the eye. The method determines the characteristic by detecting an increase in the axial length relative to an axial length measurement of the eye without a contact lens.

The detector is an optical coherence tomography (OCT) device, and the eye information describes surfaces of the eye. The method determines the characteristic by detecting an additional surface anterior to the cornea that represents the contact lens.

The detector is a camera, and the eye information describes one or more Purkinje images of the eye. The method determines the characteristic by detecting a Purkinje image resulting from the presence of a contact lens.

The eye information describes surfaces of the eye. The method determines the characteristic by detecting an additional surface anterior to the cornea.

The method performs the contact lens check automatically for each patient.

The method performs the contact lens check in response to a request to perform the contact lens check.

The method provides, by the output device comprising a display device, a visual indicator as the notification warning of the potential presence of a contact lens.

The method provides, by the output device comprises a speaker, an auditory indicator as the notification warning of the potential presence of a contact lens.

### BRIEF DESCRIPTION OF THE DRAWINGS

FIG. 1 illustrates an example of an ophthalmic system for examining an eye of a patient, according to certain embodiments;

FIGS. 2A to 2E illustrate examples of eye information that may be used to determine a characteristic indicating the potential presence of a contact lens, according to certain embodiments;

FIGS. 3A and 3B illustrate examples of a display device that provides a notification that is a visual indicator, according to certain embodiments;

FIGS. 4A and 4B illustrate examples of a speaker that provides a notification that is an auditory indicator, according to certain embodiments; and

FIG. 5 illustrates an example of a method for examining the eye of a patient that may be performed by the system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

During an eye exam, the patient may inadvertently fail to take out their contact lenses. This may yield incorrect measurements and thus faulty treatment planning and suboptimal surgical outcomes. Embodiments of the ophthalmic system described herein address this problem. The system performs a contact lens check that analyzes whether measurements indicate the potential presence of a contact lens. If they do, the system provides a notification warning of the potential presence.

Known systems do not perform such contact lens checks and instead rely on a technician to remember to ask each patient to remove any contact lens. Certain embodiments of the system automatically perform the contact lens check on each patient, so regardless of clinic workflow and technician training, the system can provide a warning if a patient is wearing contacts during the measurement. Catching these cases can prevent incorrect treatment planning and suboptimal surgical outcomes.

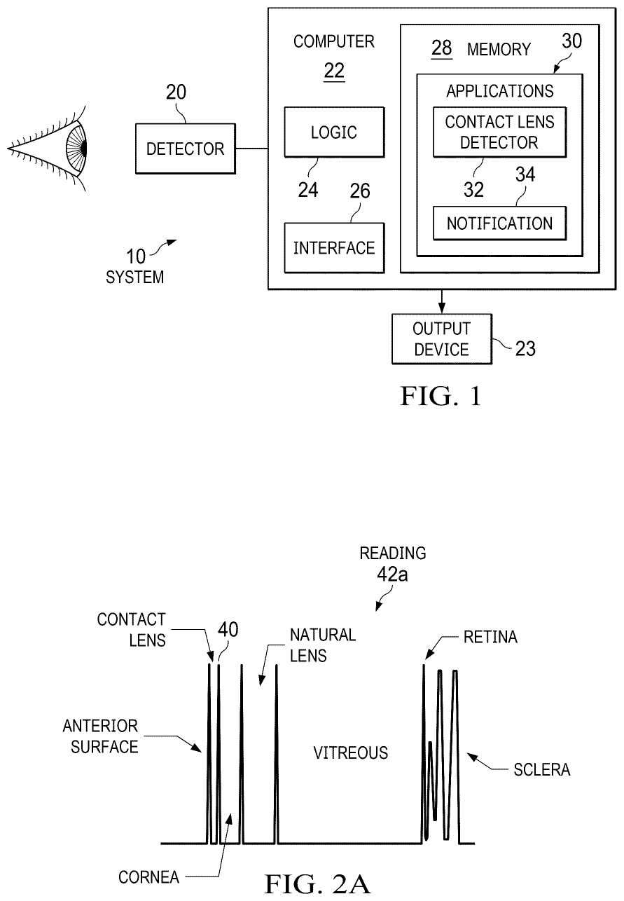

FIG. 1 illustrates an example of an ophthalmic system 10 for examining an eye of a patient, according to certain embodiments. In the example, system 10 includes a detector 20, a computer 22, and an output device 23. Computer 22 includes logic 24, an interface (IF) 26, and a memory 28, which stores applications 30 such as contact lens detector 32 and notification 34 applications.

According to an example overview, detector 20 detects waves (e.g., light or sound waves) reflected from the eye of the patient. The waves convey information about the eye, and detector 20 generates a detector signal that communicates the eye information. Computer 22 performs a contact lens check by receiving the information about the eye via the detector signal. Computer 22 determines a characteristic from the information that indicates the potential presence of a contact lens. Computer 22 generates a notification warning of the potential contact lens presence and instructs an output device 23 to provide the notification.

Turning to the example components, detector 20 detects waves (e.g., light or sound waves) reflected from the eye of the patient and generates a detector signal that communicates eye information. Detector 20 may be any suitable ophthalmic diagnostic device that can measure features of an eye. Examples of detector 20 includes an ocular biometer, keratometer, optical coherence tomography (OCT) device, camera, Scheimpflug imaging detector, wavefront detector, or any other ophthalmic diagnostic device. Eye information includes images, descriptions, and/or measurements of the eye, such as dimensions (e.g., length or anterior chamber depth), shape, density, tear film assessment, or other eye information.

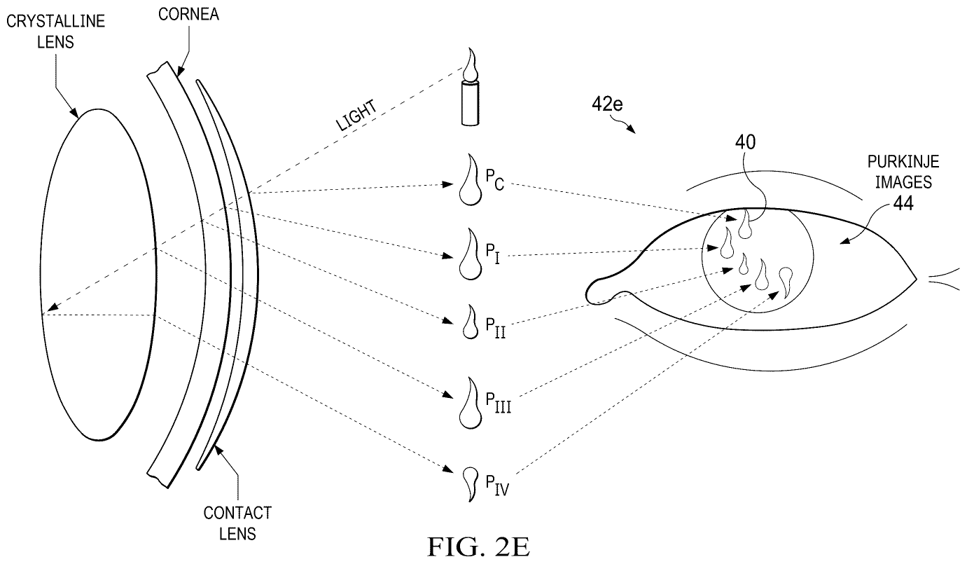

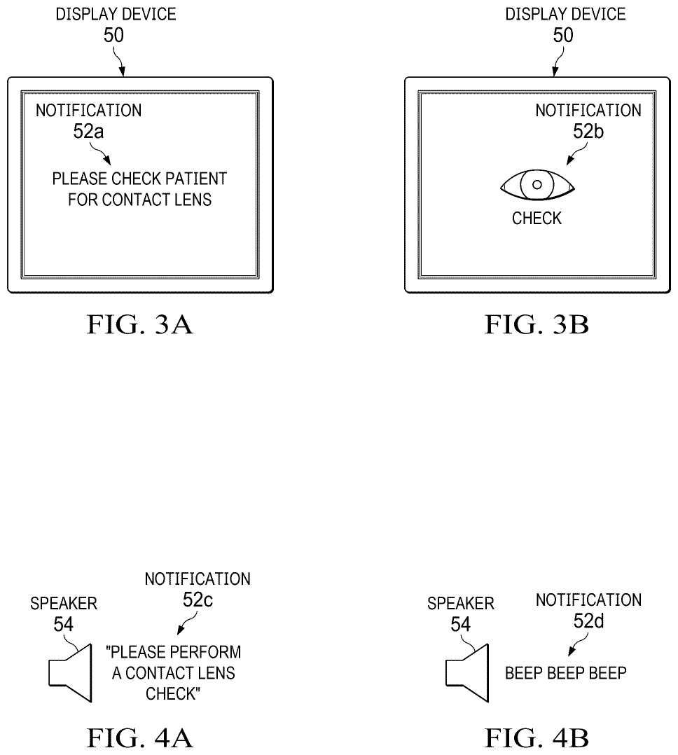

Output device 23 may be, e.g., a display device or a speaker. A display device may be a computer or display screen or monitor that can display information in a pictorial or textual form. In certain embodiments, a display device provides the notification as a visual indicator warning of the potential presence of a contact lens. A speaker can provide information in an auditory form. In certain embodiments, a speaker provides the notification as an auditory indicator warning of the potential presence of a contact lens. Output devices 23 are described in more detail with reference to FIGS. 3A to 4B.

Computer 22 sends instructions to control the components of system 10, such as detector 20 and/or output device 23, to perform a contact lens check. Computer 22 may perform the contact lens check automatically for each patient or in response to a user request to perform the check. Computer 22 uses applications 30 to perform these operations. For example, computer 22 uses contact lens detector application 32 to identify a characteristic indicating the potential presence of a contact lens. As another example, computer 22 uses notification application 34 to generate a visual or auditory notification warning of the potential presence of a contact lens.

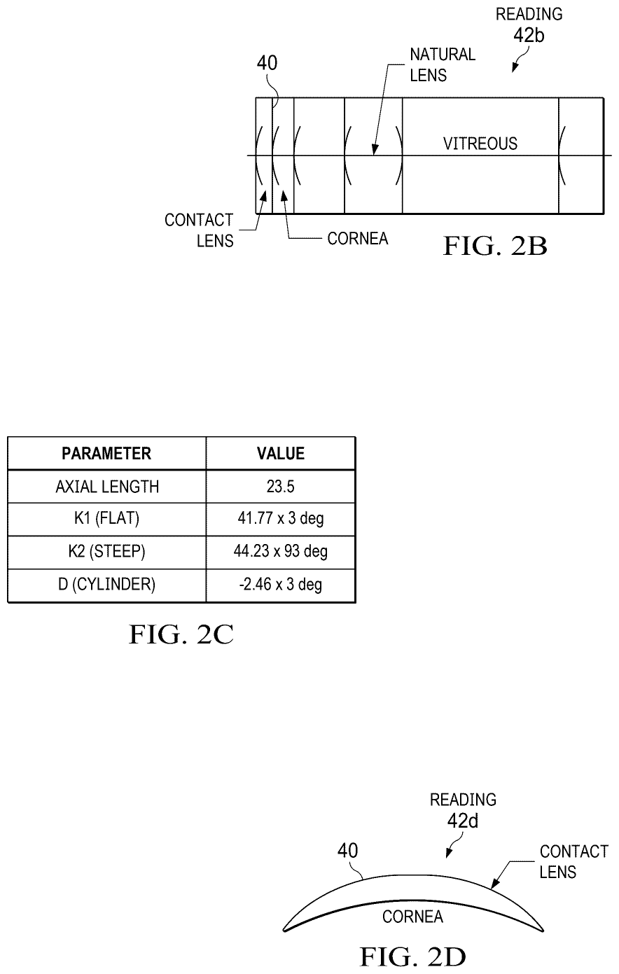

FIGS. 2A to 2E illustrate examples of eye information that may be used to determine a characteristic 40 indicating the potential presence of a contact lens, according to certain embodiments. In general, the eye information may describe the structures of the eye, such as the shape or size of features of the eye. FIGS. 2A and 2B illustrate examples of characteristics 40 found in eye information obtained from an ocular biometer. In certain embodiments, detector 20 is an ocular biometer that yields eye information describing segmentation of the anterior portion of the eye, which includes the cornea. An ocular biometer may use any suitable biometry technique, such as ultrasound biometry or optical biometry (e.g., laser partial coherence interferometry (PCI) or optical low-coherence reflectometry (OLCR)). In certain embodiments, computer 22 determines the characteristic 40 indicating the presence of a contact lens by detecting an additional segment anterior to the cornea.

FIG. 2A illustrates an example reading 42a obtained from an ultrasound ocular biometer. Ultrasound biometry uses a high-frequency sound wave generated by a probe to measure the axial length of the eye and internal structures. The sound wave travels through the eye from anterior to posterior, passing through structures such as the cornea, anterior chamber, lens, and vitreous before reaching the retina. The structures manifest as spikes on an A-scan reading 42a. In the example reading 42a, the initial spike represents the probe tip on the anterior surface of a contact lens, followed by spikes representing surfaces between the contact lens, cornea, natural lens, vitreous, retina, and sclera. The spikes representing the anterior and posterior surfaces of the contact lens can each or both be characteristics 40 indicating the presence of a contact lens.

FIG. 2B illustrates an example reading 42b obtained from an optical ocular biometer. Laser partial coherence interferometry (PCI) and optical low-coherence reflectometry (OLCR) use interference to determine the distances between the surfaces of the contact lens, cornea, natural lens, vitreous, and retina. In the example reading 42b, the vertical lines represent the anterior surface of a contact lens, followed by lines representing the surfaces between the contact lens, cornea, natural lens, vitreous, retina, and sclera. The lines representing the anterior and posterior surfaces of the contact lens can each or both be characteristics 40 indicating the presence of a contact lens.

FIG. 2C illustrates an example reading 42c obtained from a keratometer. In certain embodiments, detector 20 is a keratometer that yields a reading 42c describing the shape of the anterior portion of the eye. Reading 42c includes keratometric data, e.g., values for the axial length, K1 (flat), K2 (steep), and D (cylinder) parameters. Any suitable characteristic 40 may indicate the presence of a contact lens. For example, the contact lens adds a layer to the eye so may cause an increase in the axial length compared to a measurement of the length of the eye without the contact lens. The increase is generally the thickness of the contact lens, e.g., 50 to 150 micrometers. As another example, the contact lens adds another surface to the eye so may yield a reading that shows the additional surface.

FIG. 2D illustrates an example reading 42d obtained from a three-dimensional (3D) optical coherence tomography (OCT) device. In certain embodiments, detector 20 is an OCT device that yields reading 42d describing exterior and interior surfaces of the eye. In the embodiments, computer 22 detects an additional surface anterior to the cornea as the characteristic 40 indicating the presence of a contact lens. In the example, reading 42d shows the anterior surface of a contact lens.

FIG. 2E illustrates an example reading 42e that is an image obtained from a camera. In certain embodiments, detector 20 is a camera that can capture an image of the eye that shows one or more Purkinje images (or Purkinje reflexes) 44, which are reflections of objects from the structure of the eye. In the example, Purkinje image P_{C} is the reflection from the outer surface of the contact lens, which is the characteristic 40 indicating the presence of a contact lens. Regarding the rest of the Purkinje images: P₁ is the reflection from the anterior surface of the cornea; P₂ is from the posterior surface of the cornea; P₃ is from the outer anterior surface of the lens; and P₄ (which is inverted) is from the posterior surface of the lens.

FIGS. 3A and 3B illustrate examples of a display device 50 that provides a notification 52 (52a, 52b) that is a visual indicator, according to certain embodiments. Notification 52 may be any suitable visual indicator. In the examples, notification 52a comprises text, e.g., “Please check the patient for a contact lens.” Notification 52b is a graphic that may be different colors depending on the notification. For example, if the graphic is red, the technician should check the patient for, e.g., a contact lens. The graphic may turn yellow when system 10 is analyzing readings from detector 20. If system 10 does not detect any issues (e.g., the presence of a contact lens), the graphic may turn green indicating there are no detectable problems.

FIGS. 4A and 4B illustrate examples of a speaker 54 that provides a notification 52 (52c, 52d) that is an auditory indicator, according to certain embodiments. Notification 52 may be any suitable auditory indicator. In the examples, notification 52c comprises a speech warning of the potential presence of a contact lens. Notification 52b is a beeping noise indicating the potential presence of a contact lens.

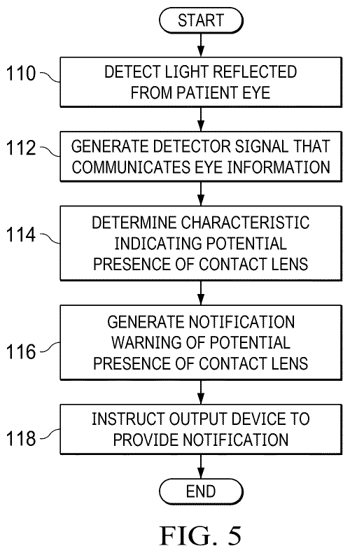

FIG. 5 illustrates an example of a method for examining the eye of a patient that may be performed by system 10 of FIG. 1, according to certain embodiments. In the example, the method starts at step 110, where detector 20 detects waves (e.g., light or sound waves) received from the eye. Detector 20 may be, e.g., a biometer, keratometer, OCT device, and/or any other suitable ophthalmic diagnostic device. At step 112, detector 20 generates a detector signal that communicates eye information conveyed by the waves.

Computer 22 performs a contact lens check at steps 114 to 118. Computer 22 may perform the contact lens check automatically for each patient and/or may perform the contact lens check in response to a user request to perform the contact lens check. At step 114, computer 22 determines a characteristic indicating the potential presence of a contact lens from the eye information. For example, computer 22 may detect an additional segment, surface, or shape anterior to the cornea or may detect a measurement (e.g., an increased axial length) indicating the presence of a contact lens.

Computer 22 generates a notification warning of the potential presence of a contact lens at step 116 and instructs output device 23 to provide the notification at step 118. In certain embodiments, the output device 23 is a display device 50, and the notification is a visual indicator warning of the potential presence of a contact lens. In certain embodiments, output device 23 is a speaker 54, and the notification is an auditory indicator warning of the potential presence of a contact lens.

A component (such as the control computer) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112 (f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112 (f).

What is claimed:

1. An ophthalmic system for examining an eye of a patient, comprising:
a detector configured to:
detect a plurality of waves reflected from the eye of the patient, the waves conveying information about the eye; and
generate a detector signal that communicates the information about the eye; and
a computer configured to perform a contact lens check by:
receiving the information about the eye via the detector signal;
determining from the information about the eye a characteristic indicating a potential presence of a contact lens;
generating a notification warning of the potential presence of a contact lens; and
instructing an output device to provide the notification warning of the potential presence of a contact lens.

2. The ophthalmic system of claim 1, wherein:
the detector comprises an ocular biometer;
the information about the eye describes a segmentation of an anterior portion of the eye, the anterior portion comprising a cornea; and
the computer is configured to determine the characteristic indicating the presence of a contact lens by:
detecting a segment anterior to the cornea, the segment representing the contact lens.

3. The ophthalmic system of claim 1, wherein:
the detector comprises a keratometer;
the information about the eye describes an axial length of the eye; and
the computer is configured to determine the characteristic indicating the presence of a contact lens by:
detecting an increase in the axial length relative to an axial length measurement of the eye without a contact lens.

4. The ophthalmic system of claim 1, wherein:
the detector comprises an optical coherence tomography (OCT) device;
the information about the eye describes a plurality of surfaces of the eye; and
the computer is configured to determine the characteristic indicating the presence of a contact lens by:
detecting a surface anterior to the cornea, the surface representing the contact lens.

5. The ophthalmic system of claim 1, wherein:
the detector comprises a camera;
the information about the eye describes one or more Purkinje images of the eye; and
the computer is configured to determine the characteristic indicating the presence of a contact lens by:
detecting a Purkinje image resulting from the contact lens.

6. The ophthalmic system of claim 1, wherein:
the information about the eye describes a plurality of surfaces of the eye; and
the computer is configured to determine the characteristic indicating the presence of a contact lens by:
detecting a surface anterior to the cornea, the surface representing the contact lens.

7. The ophthalmic system of claim 1, the computer configured to perform the contact lens check automatically for each patient.

8. The ophthalmic system of claim 1, the computer configured to perform the contact lens check in response to a request to perform the contact lens check.

9. The ophthalmic system of claim 1, wherein:
the output device comprises a display device; and
the notification comprises a visual indicator warning of the potential presence of a contact lens.

10. The ophthalmic system of claim 1, wherein:
the output device comprises a speaker; and
the notification comprises an auditory indicator warning of the potential presence of a contact lens.

11. A method for examining an eye of a patient, comprising:
detecting, by a detector, a plurality of waves reflected from the eye of the patient, the waves conveying information about the eye;
generating, by the detector, a detector signal that communicates the information about the eye; and
performing, by a computer, a contact lens check by:
receiving the information about the eye via the detector signal;
determining from the information about the eye a characteristic indicating a potential presence of a contact lens;
generating a notification warning of the potential presence of a contact lens; and
instructing an output device to provide the notification warning of the potential presence of a contact lens.

12. The method of claim 11, wherein:
the detector comprises an ocular biometer;
the information about the eye describes a segmentation of an anterior portion of the eye, the anterior portion comprising the cornea; and
determining the characteristic indicating the presence of a contact lens comprises detecting a segment anterior to the cornea, the segment representing the contact lens.

13. The method of claim 11, wherein:
the detector comprises a keratometer;
the information about the eye describes an axial length of the eye; and
determining the characteristic indicating the presence of a contact lens comprises detecting an increase in the axial length relative to an axial length measurement of the eye without a contact lens.

14. The method of claim 11, wherein:
the detector comprises an optical coherence tomography (OCT) device;
the information about the eye describes a plurality of surfaces of the eye; and
determining the characteristic indicating the presence of a contact lens comprises detecting a surface anterior to the cornea, the surface representing the contact lens.

15. The method of claim 11, wherein:
the detector comprises a camera;
the information about the eye describes one or more Purkinje images of the eye; and
determining the characteristic indicating the presence of a contact lens comprises detecting a Purkinje image resulting from the presence of a contact lens.

16. The method of claim 11, wherein:
the information about the eye describes a plurality of surfaces of the eye; and
determining the characteristic indicating the presence of a contact lens comprises detecting a surface anterior to the cornea, the surface representing the contact lens.

17. The method of claim 11, further comprising:
performing the contact lens check automatically for each patient.

18. The method of claim 11, further comprising:
performing the contact lens check in response to a request to perform the contact lens check.

19. The method of claim 11, further comprising:
providing, by the output device comprising a display device, a visual indicator as the notification warning of the potential presence of a contact lens.

20. The method of claim 11, further comprising:
providing, by the output device comprises a speaker, an auditory indicator as the notification warning of the potential presence of a contact lens.
